(19) United States
(12) Patent Application Publication
(10) Pub. No.: US 2022/0118290 A1
(43) Pub. Date: Apr. 21, 2022

(54) POWERED AIR FILTRATION FACE COVERING

(71) Applicant: DRS Innovations LLC, Pasadena, CA (US)

(72) Inventors: Grant Verdun Delgatty, La Canada Flintridge, CA (US); Safir Bellali, Pasadena, CA (US)

(21) Appl. No.: 17/506,010

(22) Filed: Oct. 20, 2021

Related U.S. Application Data
(60) Provisional application No. 63/094,656, filed on Oct. 21, 2020.

(51) Int. Cl. A62B 18/00; A61F 9/02; A62B 18/08; A62B 7/10; A61L 9/20; A41D 13/11

(52) U.S. Cl. CPC A62B 18/003; A61F 9/029; A62B 18/084; A62B 7/10; A61L 2209/12; A41D 13/1184; A61L 2209/14; A61L 2209/15; A61L 9/20

(57) ABSTRACT

A protective face covering may comprise a frame comprising a main body. At least a portion of the main body is hollow and defines an inlet passage and an exhaust passage therein. The protective face covering may further comprise a visor coupled to the frame and configured to be disposed adjacent a face of a user in a first position, a membrane barrier coupled the visor and configured to seal against a portion of the wearer to define a sealed cavity between the visor and the face of the wearer in the first position, a blower unit in fluid communication with the inlet passage, the blower unit configured to provide a volume of filtered fluid to the inlet passage. The frame comprises an inlet aperture disposed adjacent a front portion of the frame and configured to direct a curtain of fluid into the sealed cavity in the first position.

### CROSS-REFERENCE TO RELATED APPLICATIONS

This application is a non-provisional of and claims the benefit of U.S. Provisional Application No. 63/094,656 filed on Oct. 21, 2020, which is hereby incorporated by reference in its entirety.

### BACKGROUND

Face coverings may be used for filtering and protecting the wearer from airborne particulates such as dust, mold spores, pollution and viruses. Conventional face coverings have taken many forms, often dictated by the environment and context of use. Some face coverings are designed for high levels of filtration, often utilizing filter materials such as HEPA (High Efficiency Particulate Arrestance) and/or N95 or higher rated material suitable for blocking microscopic particulate matter.

Other face coverings utilize certain fabric materials such as cloth, paper, polypropylene fiber, etc. to cover the nose and mouth which, although they provide a certain level of protection, are not designed to protect the wearer from breathing in very small particulate matter.

Other face coverings, often referred to as PAPR (Powered Air Purification Respirator) devices, offer an even higher level of protection and comfort against aerosolized particulate matter by encapsulating the wearer's nose, mouth and eyes in a contained cavity.

However, improvements are needed.

### SUMMARY

The disclosure relates to face coverings and methods of filtering fluids such as air. As an example, a protective face covering may comprise a frame comprising a main body, wherein at least a portion of the main body is hollow and defines an inlet passage and an exhaust passage therein, a visor coupled to the frame and configured to be disposed adjacent a face of a user in a first position, a membrane barrier coupled the visor and configured to seal against a portion of the wearer to define a sealed cavity between the visor and the face of the wearer in the first position, a blower unit in fluid communication with the inlet passage, the blower unit configured to provide a volume of filtered fluid to the inlet passage, wherein the frame comprises an inlet aperture disposed adjacent a front portion of the frame and configured to direct a curtain of fluid into the sealed cavity in the first position.

A powered air filtration face covering may comprise an electrically powered air filtration blower unit which is designed to force ambient air through a filter to remove the majority of particulate and/or viral matter from the surrounding environment. The clean filtered air is transferred through a single or plurality of flexible hoses or other means of distribution and is delivered to a face covering device by where the wearer is able to see and be seen through an uninhibited clear lens which is designed to protect the wearer's entire face including the mouth, nose and eyes. The clear lens is sealed to the perimeter of the wearer's face with an airtight flexible membrane barrier, by where the positive air pressure continuously filling the interior cavity space of the device is exhaled through an exhaust port on the device. The exhausted air leaving the device is also forced through a filter, allowing the exhausted air leaving the device to be restricted from emitting any potential viral matter being exhaled by the wearer. The device is also designed to be manipulated to allow the wearer to gain access to his or her face while still wearing the device, while continuing to maintain an elevated level of protection against outside particulate or viral matter.

One or more examples and aspects described herein relate to face coverings that may be used for complete mouth, nose and eye protection against exposure to particulate and viral matter, clear visibility of the wearer's face with the convenience of unobstructed breathing, continued protection while the user is able to access his or her face, filtration of the user's exhausted air, ease of user experience in donning and doffing the device,

reduction of parts and complexity to achieve a less obtrusive form factor while having the same or higher level of protection as more complex products, and processes of making and using such products and devices.

### BRIEF DESCRIPTION OF THE DRAWINGS

The above and other aspects and features of the present invention will become more apparent to those skilled in the art from the following detailed description of the example embodiments with reference to the accompanying drawings, in which:

FIG. 1 is an overhead front-right-side perspective view of the combination of components found in an example powered air filtration face covering device according to embodiments of the disclosure.

FIG. 2 is a front-right-side perspective view of an electrically powered air filtration blower unit according to embodiments of the disclosure.

FIG. 3 is perspective view of an electrically powered air filtration blower unit according to embodiments of the disclosure.

FIG. 4 is a below front-right-side perspective view of an example hollow air frame according to embodiments of the disclosure.

FIG. 5 is an overhead front-right-side perspective view of an example visor assembly according to embodiments of the disclosure.

FIG. 6 is a left-side view showing a visor assembly according to embodiments of the disclosure.

FIG. 7 is a left-side view showing a visor assembly according to embodiments of the disclosure.

FIG. 8 is a detailed illustration of an example visor assembly and head strap connection point according to embodiments of the disclosure.

FIG. 9 is a rear-left-side perspective view of an example visor assembly, head strap, elastic loop connector, and flexible air hose according to embodiments of the disclosure.

FIG. 10 is an exploded view of a hollow air frame according to embodiments of the disclosure.

FIG. 11 is a rear-left-side perspective view of an alternative configuration for the powered filter blower unit of FIG. 2 according to embodiments of the disclosure.

FIG. 12 is a front-left-side perspective view of a powered air filtration face covering device according to embodiments of the disclosure being utilized in an application.

FIG. 13 illustrates a cross-section of an example coupling mechanism for overhead air receptacles commonly found in airplanes.

### DETAILED DESCRIPTION

Hereinafter, example embodiments will be described in more detail with reference to the accompanying drawings. The present disclosure, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof may not be repeated. Further, features or aspects within each example embodiment should typically be considered as available for other similar features or aspects in other example embodiments.

In the drawings, the relative sizes of elements, layers, and regions may be exaggerated and/or simplified for clarity. Spatially relative terms, such as “beneath,” “below,” “lower,” “under,” “above,” “upper,” “front” and “rear” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

It will be understood that when an element or layer is referred to as being “on,” “connected to,” or “coupled to” another element or layer, it can be directly on, connected to, or coupled to the other element or layer, or one or more intervening elements or layers may be present. In addition, it will also be understood that when an element or layer is referred to as being “between” two elements or layers, it can be the only element or layer between the two elements or layers, or one or more intervening elements or layers may also be present

The terminology used herein is for the purpose of describing particular embodiments and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” “has,” “have,” and “having,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

As used herein, the term “substantially,” “about,” and similar terms are used as terms of approximation and not as terms of degree, and are intended to account for the inherent variations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively. Also, the term “exemplary” is intended to refer to an example or illustration.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Conventionally, most filtration face coverings or masks only protect the wearer from breathing in outside particulate matter and do not filter exhausted air coming from the wearer's exhaled air. Many of the conventional products only protect the wearer from breathing in particulate matter and do not protect the wearer's eyes from exposure to said particles. Additionally, most ‘face masks’ are restricted to being made from breathable and opaque textile materials. As such, these products have a tendency to muffle the wearer's speech, and do not allow the wearer's face/mouth to be seen by others, causing difficulty in normal communication.

Additionally, PAPR devices are often designed to filter air coming into the cavity of the device, and do not provide filtered exhaust air, accounting for the potential of exhausted air coming from the wearer's own exhalation being contaminated. Additionally, if the filter is exposed to heavy volumes of particulate or viral matter, the filter may be required to be replaced to maintain efficacy. As such, the wearer of the device would need to minimize exposure of particulate or viral matter if using the device while replacing the filter.

Additionally, in some environmental applications, such as airline travel or other close quarter environments, individuals are required to sit or stand in relatively close proximity to one another, allowing the air space around them to become exposed to potential aerosolized viral organisms being transferred between individuals.

The above comments relate to some of the limitations of current products and devices commonly used for providing protection against breathing in and exhausting particulate and viral matter. Example embodiments of the present disclosure are described herein and may include examples configured to address some or all of the limitations discussed above. Other examples are configured for providing other functions and improvements. The above information disclosed in this section is for enhancement of understanding of the background of the disclosure, and therefore, it may contain information that does not constitute prior art.

Example embodiments described herein relate to powered filtered air face coverings and components thereof.

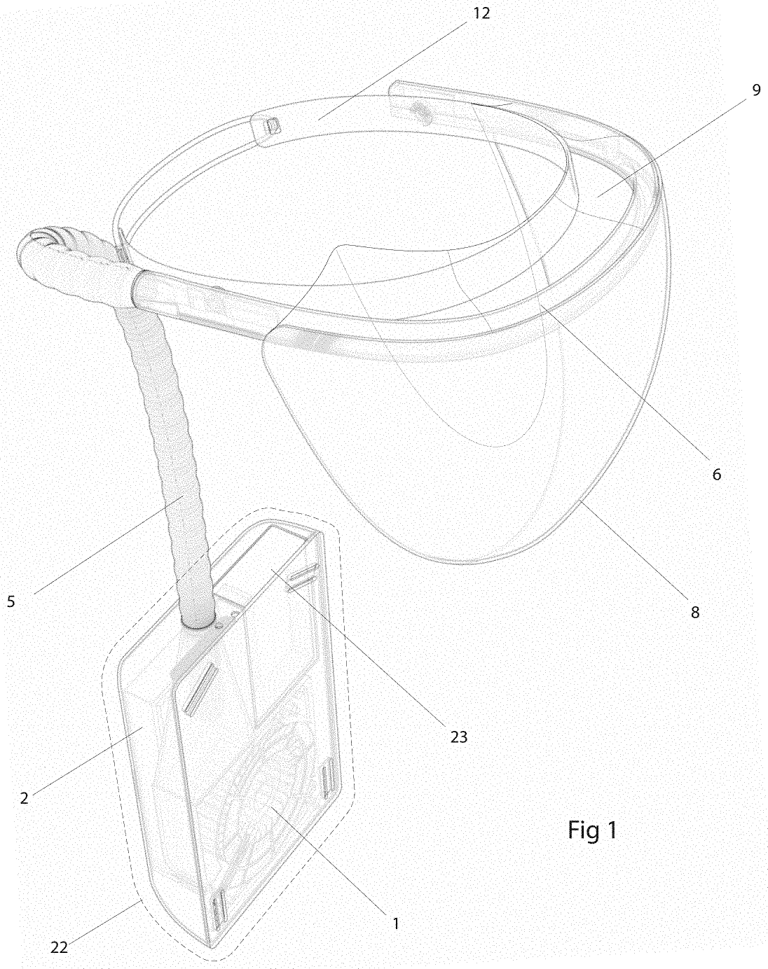

According to embodiments of the disclosure, a protective face covering is disclosed. FIG. 1 is an overhead front-right-side perspective view of an exemplary combination of components found in an example powered air filtration protective face covering device according to embodiments of the disclosure. The protective face covering may comprise a frame 6 configured to be mounted on a head of a wearer. The frame 6 may comprise a main body. The main body may have a curvilinear shape configured to extend around a forehead of the wearer.

The main body may have a curvilinear shape configured to extend around a forehead of the wearer. At least a portion of the main body may be hollow. At least a portion of the main body may define an inlet passage configured to receive and direct fluid such as filtered air toward a face of a wearer. At least a portion of the main body may define an exhaust passage configured to receive and direct fluid away from the face of a wearer. Each of the inlet passage and the exhaust passage may comprise a respective inlet and outlet to/from the passage itself, but reference may be made as to the inlet of fluid toward the face or area around the face or visor of the face covering and the exhaust of fluid away from the face or area around the face. As described herein, the flow of fluid toward and away from the face creates a fresh cycle of fluid such as air that minimizes a wearer's inhaling of their own exhaled air. In an aspect, a wearer may inhale fresh, filtered air provided by the inlet passage, while their exhaled air is moved away and exhausted through the exhaust passage.

The frame may also comprise an inlet aperture disposed adjacent a front portion of the frame and configured to direct a curtain of fluid into the sealed cavity in the first position. A curtain of filtered air may be amount of air blown with sufficient force to effectively form a “wall” of air such that particulate or viral matter in the environment would be prevented from penetrating the wall and would instead be blown in the direction of the air, thereby protecting a wearer from inhaling the particulate or viral matter. For instance, the inlet aperture may be configured to direct a curtain of fluid across the face of the wearer. The protective face covering may further comprise an internal dam wall disposed inside the inlet passage and adjacent the inlet aperture. The internal dam wall may operate to direct forced air to flow through the inlet aperture (which may be a respective outlet from the inlet passage) to an area around the face of a wearer. The internal dam wall may operate to direct forced air to flow through the inlet aperture, through the sealed cavity, and to the exhaust passage. Each of the inlet passage and the exhaust passage may comprise a respective inlet and outlet to/from the passage itself, but reference may be made as to the inlet of fluid toward the face or area around the face or visor of the face covering and the exhaust of fluid away from the face or area around the face. As described herein, the flow of fluid toward and away from the face creates a fresh cycle of fluid such as air that minimizes a wearer's inhaling of their own exhaled air. In an aspect, a wearer may inhale fresh, filtered air provided by the inlet passage, while their exhaled air is moved away and exhausted through the exhaust passage.

The protective face covering may include a visor. The visor may be co-molded with the frame or may be separate and coupled thereto. The visor may be disposed adjacent a front portion of the frame. The visor may be coupled to the frame and may be configured to be disposed adjacent a face of a user in a first position. The visor may be configured to be disposed adjacent a face of a user in a first position such as a closed position. The visor may be configured to move or rotate between a plurality of positions relative to a face a wearer. In an aspect, various positions of the visor may be configured by using a mechanism to secure the visor in a position relative to a wearer's face, mouth, or nose. The visor may be rotatably coupled to the frame and configured to be disposed adjacent a face of a user in a closed position and rotated about the frame into a second position. The visor may be configured to rotationally pivot to one or more positions. The visor may be configured to rotationally pivot to a second position without impeding the curtain of fluid.

A membrane barrier may be coupled to one or more of the visor or the frame and configured to seal against a portion of the wearer to define a sealed cavity between the visor and the face of the wearer. A membrane barrier may be coupled to one or more of the visor or the frame. The membrane barrier may be configured to seal against a portion of the wearer to define a sealed cavity between the visor and the face of the wearer in the first position. The membrane barrier may be configured to contact a portion of a wearer's head or face to provide a fluid barrier. In the first position, the membrane barrier may extend around a face of the wearer. In a second position, the membrane barrier may be spaced from a face of the wearer. In a third position, the membrane barrier may be disposed between a mouth and nose of a wearer such that the nose is disposed in a sealed cavity defined by the membrane barrier and the visor and the mouth is open to the ambient environment. Various positions of the visor and the membrane barrier may be used. As a non-limiting example, the membrane barrier may comprise or may be formed from polyurethane (PU). Other materials may be used or in combination, such as vinyl, ethylene-vinyl acetate (EVA), rubber, silicone, polyethylene (PE), polypropylene (PP), polystyrene (PS), polychloroprene, and the like.

As the visor is rotated, the membrane barrier may contact various parts of the head or face of the wearer to create a sealed cavity defined by the membrane barrier and the visor. However, as the visor is rotated, the membrane barrier may space from the wearer. In certain aspects, regardless of the visor position, the inlet passage may be configured to provide a curtain of filtered air that is forced toward or across a face of the user. As such, the curtain of air provides fresh, filtered, air to a wearer, even if the membrane barrier is not sealed against the wearer.

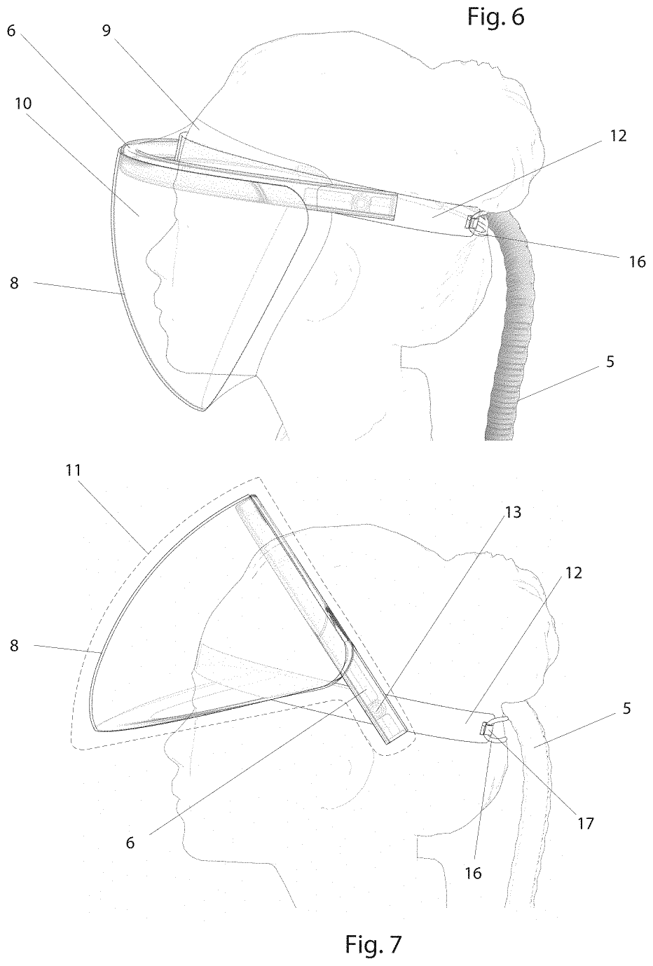

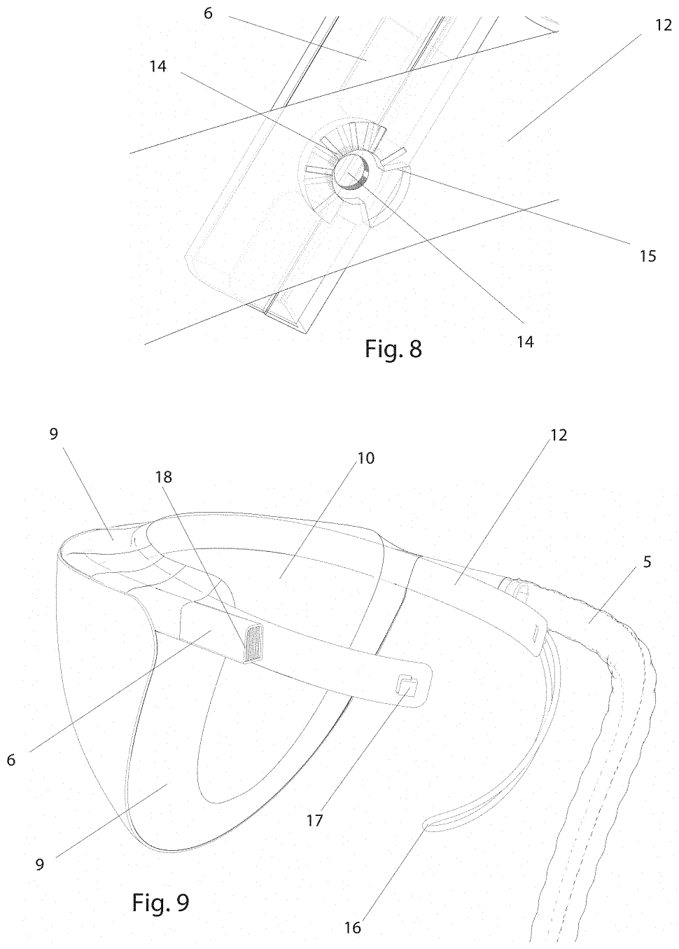

The protective face covering may include a visor assembly. FIG. 5 is an overhead front-right-side perspective view of an example visor assembly 11 according to embodiments of the disclosure, which may be similar to the visor shown FIG. 1, and may include a clear lens 8, a hollow air frame 6, a head strap 12, a membrane barrier 9, or a flexible air tube 5, or combination thereof. The visor assembly 11 may be disposed adjacent or may extend from the frame. FIG. 6 is a left-side view showing a visor assembly 11 according to embodiments of the disclosure, such as the visor assembly 11 shown in FIG. 5, being worn in the normal ‘closed’ position, where the wearer's face would be encapsulated within the sealed cavity of the visor assembly. FIG. 7 is a left-side view showing a visor assembly according to embodiments of the disclosure, such as the visor assembly of FIG. 6 being worn in the rotated ‘open’ position, where the wearer's face would be accessible. FIG. 8 is a detailed illustration of an example visor assembly and head strap connection point according to embodiments of the disclosure, which depicts the rotational pivot mechanism, the rotational barrier walls, and positive engagement locking mechanism. FIG. 9 is a rear-left-side perspective view of an example visor assembly, head strap, elastic loop connector, and flexible air hose according to embodiments of the disclosure, and depicts the exhaust area of the hollow air frame, as well as the elastic loop disconnected from the snap fit hook connector on the head strap.

In certain examples, a clear lens 8 which may be molded to curve around the contours of a standard human face, would be secured to or molded in combination with the hollow air frame 6. In particular examples, a flexible elastomeric airtight membrane barrier 9 may be adhered/mounted to a peripheral or perimeter edge of the clear lens 8 and would continuously wrap onto the top surface of the hollow air frame 6 (as shown in FIG. 5), thus creating a mostly airtight continuous barrier around the wearer's face. As a non-limiting example, the membrane barrier may comprise or may be formed from polyurethane (PU). Other materials may be used or in combination, such as vinyl, ethylene-vinyl acetate (EVA), rubber, silicone, polyethylene (PE), polypropylene (PP), polystyrene (PS), polychloroprene, and the like. The membrane barrier 9 may produce a restricted cavity space 10 (as shown in FIG. 6) between the clear lens 8 and the wearer's mouth, nose and eyes, where filtered air entering the cavity space 10 through the hollow air frame 6, may become trapped, creating constant positive air pressure of clean, filtered air for the wearer to breathe freely and unobstructed from any material coverings directly in contact with the wearer's face.

In particular examples, the combination of the clear lens 8, the membrane barrier 9 and the hollow air frame 6, herein after referred to as the visor assembly 11, may be mounted to a head strap 12 (as shown in FIG. 7).

The connection point of the visor assembly 11 and the head strap 12 may include a pivot mechanism 13, by where the visor assembly 11 may be secured and pivoted in an upward motion (as shown in FIG. 7). The pivot mechanism 13 connecting the visor assembly 11 and the head strap 12 may include a definitive rotational starting and stopping point (as shown in FIG. 7), by creating rotational barrier walls 14 incorporated into the pivot mechanism 13. By allowing the visor assembly 11 to rotate in an upwards motion, the wearer is able to access his or her face (as shown in FIG. 7), while substantially still having a curtain of clean, filtered air passing over the wearer's face, allowing the wearer continued substantial protection against breathing in particulate matter from the surrounding environment. In a more specific application, the visor assembly 11 can be rotated upwards where the location of the elastomeric membrane barrier 9 can be positioned between the nose and mouth of the wearer, thus allowing the wearer to continue breathing filtered air through his or her nose while having mouth accessibility for activities such as eating and drinking. A positive engagement locking mechanism 15, may allow the visor assembly 11 to stay engaged in multiple ‘open’ positions, thus allowing the wearer to have ‘hands free’ access to his or her face while wearing the claimed device.

In certain examples, the head strap 12 may be affixed to the wearer's head by incorporating a fastener connecting the two rearward ends of the head strap 12 with an adjustable connector (as shown in FIG. 9). In particular examples, this connector may be, but not limited to, a standard elastic hair band or ‘loop’ 16 or alternative closure strap to snap into hook connectors 17 incorporated into the head strap 12 (as shown in FIG. 9). Integrating a standard elastic band/loop 16 allows the head strap 12 to have a variable, comfortable and secure fit on the wearer's head. The hook connectors 17 which anchor the elastic loop 16 to the head strap 12, allows for the wearer to easily secure the device to his or her head with a singular hand motion. Engaging different sized elastic loops 16 or alternative closure straps may allow for variable head size and shape applications of securing the head strap 12 to the wearer's head.

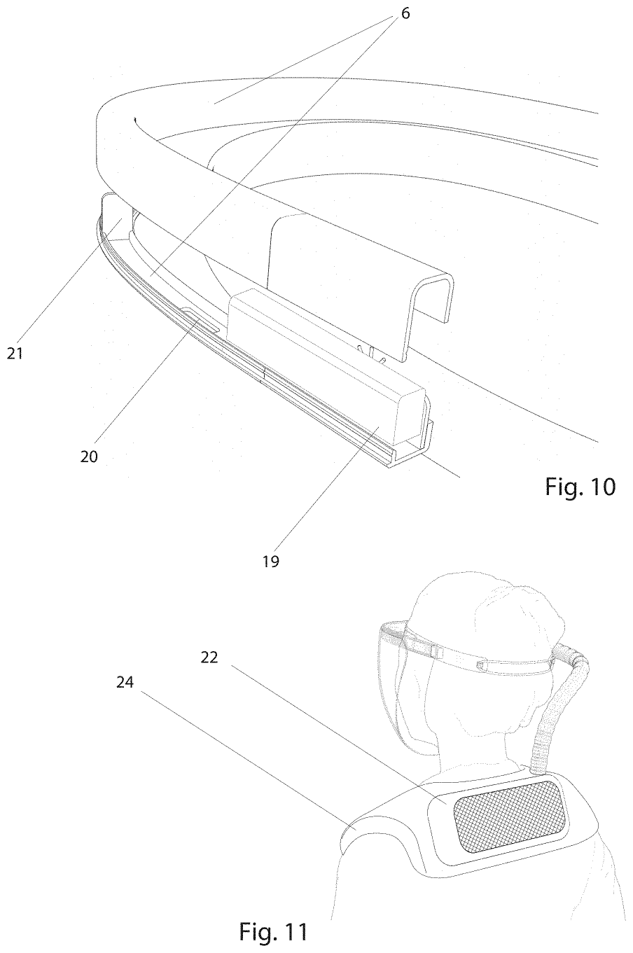

In particular examples, the air contained inside the cavity space 10 of the visor assembly 11 will become a combination of filtered clean air and exhaled air coming from the wearer's breathing. As such, the positive pressure of this encapsulated air may be forced to evacuate through an exhaust area 18 of the visor assembly 11 (as shown in FIG. 9). The protective face covering may further comprise an exhaust filter 19 disposed in the exhaust passage and configured to filter fluid passing from the sealed cavity through the exhaust passage. In order to substantially protect others from potential viral contamination produced by the wearer's exhaled breathing, the claimed device may incorporate a secondary exhaust filter 19 housed within the exhaust area 18 of the hollow air frame 6 (as shown in FIG. 9). The exhaled air will travel from the cavity space 10 through one or more exhaust intake holes 20 into the exhaust area of the frame and pushed through the exhaust filter 19 before being expelled (as shown in FIG. 10). A wall or air dam 21 inside hollow air frame 6 separates the incoming and exhaust areas of the hollow air frame 6.

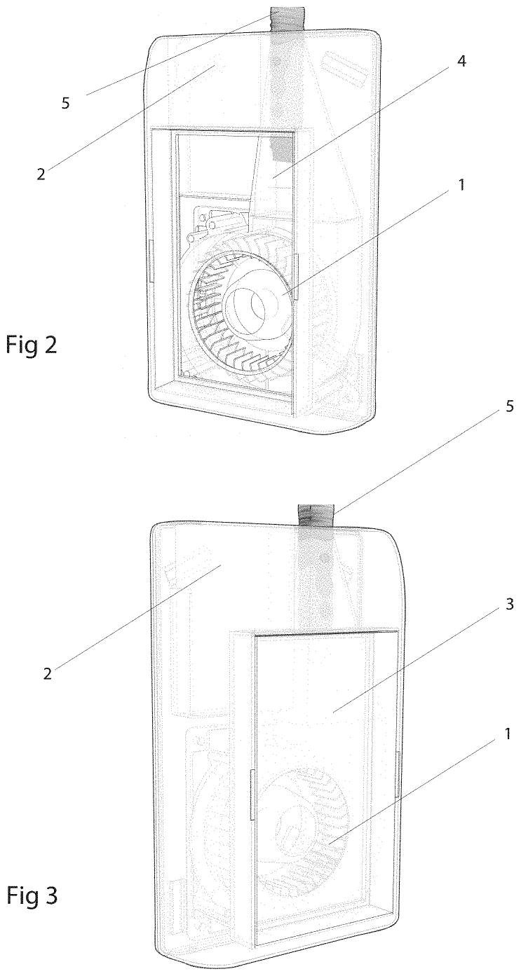

The protective face covering may include an electrically powered air filtration blower unit, and an air hose connecting the two. FIG. 2 is a front-right-side perspective view of an electrically powered air filtration blower unit according to embodiments of the disclosure, such as is shown in FIG. 1 and depicts the framed cavity area where the HEPA filter or similar would go. The blower unit may be in fluid communication with the inlet passage. The blower unit may be configured to provide a volume of filtered fluid to the inlet passage. As an example, the frame may comprise an inlet aperture disposed adjacent a front portion of the frame and configured to direct a curtain of fluid into the sealed cavity in the first position. The blower unit may be a moveable unit that may be coupled to a wearer. The blower unit may be comprised as part of a separate system from the protective face covering, such as, for example an air handling system, an airplane air system, a transit air system, or the like. As an example, the blower unit may be a powered unit. As a further example, the blower unit may be powered by one or more batteries. The blower unit may comprise a HEPA filter configured to filter fluid being provided to the inlet passage.

FIG. 2 also shows the internal components of the filter unit including the electrically powered fan/blower, the funnel area, and the removable/swappable rechargeable battery. FIG. 3 is perspective view of an electrically powered air filtration blower unit according to embodiments of the disclosure, such as is shown in FIGS. 1 and 2, which depicts the removable/swappable HEPA filter or similar in position. In particular examples, an electrically powered fan or blower 1 contained within an enclosed housing 2 draws ambient air in through a HEPA filter (or similar) 3. The air passing through the HEPA filter removes particulate matter from the ambient air, and the now filtered air is drawn into the electrically powered fan/blower. The protective face covering may further comprise a fluid conduit 5 extending between the blower unit and the frame. The fluid conduit 5 may be flexible such as a hollow air tube. The exhausted air leaving the fan/blower is then forced into a funnel area 4 and is directed into one or more flexible conduits (e.g., air tubes) 5. One or more treatment elements may be disposed in, along, or adjacent the fluid conduit 5 in order to sanitize or treat the fluid passing through the fluid conduit 5. As an example, UV-C elements such as diodes or light chips may be positioned to irradiate fluid with UV-C spectrum (e.g., 100 nm-280 nm) light. As such, the UV-C spectrum may treat the fluid. Any number and placement of the treatment elements may be used. Treatment elements may comprise light or radiation emitting elements such as those configured to emit light in the UV band (e.g., 10-400 nanometers or 100 to 380 nm), which may be subdivided into a number of ranges in accordance with ISO-21348 or UV-A (320 to 400 nm), UV-B (280 to 320 nm), and UV-C (100 to 280 nm). Other ranges may be used. FIG. 11 is a rear-left-side perspective view of an alternative configuration for the powered filtration blower unit of FIG. 2 according to embodiments of the disclosure, showing an example of how the filter unit can be worn in a variety of configurations and supported by a variety of body mounting contraptions. In this particular depiction, the filtration blower unit is being worn over the wearer's shoulders.

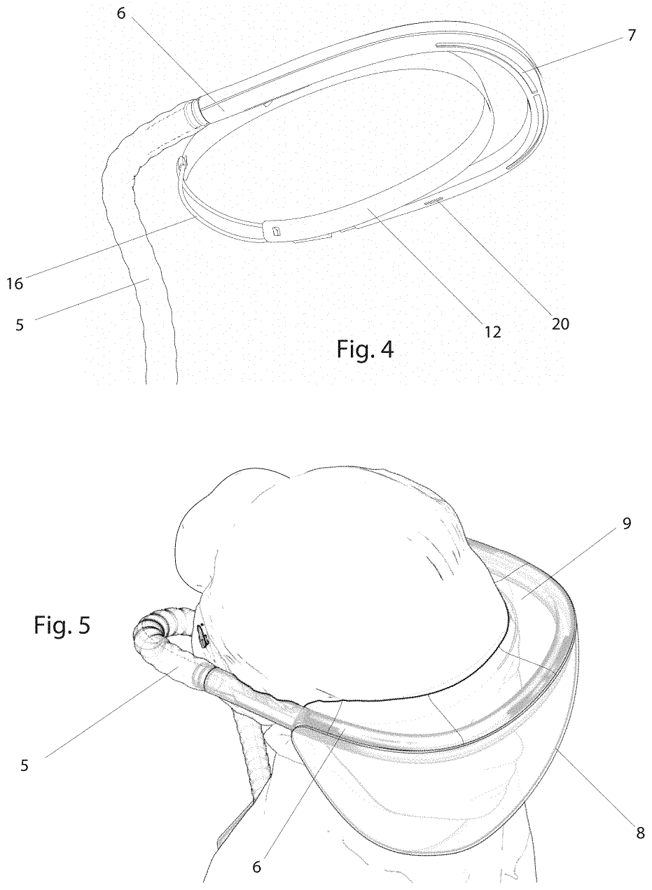

FIG. 4 is a below front-right-side perspective view of an example hollow air frame 6 according to embodiments of the disclosure, including a head strap, an elastic loop connector, and a flexible air tube. In particular examples, the filtered air traveling in the flexible air tube(s) 5 will enter into a hollow air frame 6 which is curved to fit around the frontal forehead portion of a human head. The hollow air frame 6 incorporates an opening slit 7 around the lower perimeter edge of the frontal portion of the hollow air frame 6, in which air entering the frame from the flexible air tube(s) 5 is exhausted through the opening slit in a downward direction creating a curtain of filtered air in front of the user's face. FIG. 10 is an exploded view of a hollow air frame according to embodiments of the disclosure, such as the hollow air frame of FIG. 4. FIG. 4 further depicts how the frame may be separated into two parts (top and bottom), for example, and depicts an exhaust filter, an internal wall/air dam, and an exhaust intake hole.

In certain examples, the housing 2 of the electrically powered air filtration blower unit 22 may house a rechargeable battery 23 which may be removable/swappable in order to allow the filtration blower unit 22 to run continuously with minimal disruption by quickly swapping battery units. In addition, the filtration blower unit 22 may contain two or more removable/swappable rechargeable batteries 23, allowing the filtration blower unit 22 to run continuously without disruption when swapping battery units.

In particular examples, the housing 2 of the electrically powered air filtration blower unit 22 may house a HEPA filter or similar 3 which may be removable/swappable in order to allow the filtration blower unit 22 to maintain efficacy with minimal disruption by quickly swapping filters. In addition, the filtration blower unit 22 may contain two or more removable/swappable HEPA filters or similar 3, allowing the filtration blower unit 22 to continuously maintain efficacy without disruption when swapping filters.

In certain examples, the portable filtration system may be embedded into a variety of wearable contraptions or assemblies 24 that can be worn comfortably and securely under different circumstances, including, but not limited to on the shoulders, hips, back, chest, leg with or without straps (as shown in FIG. 11).

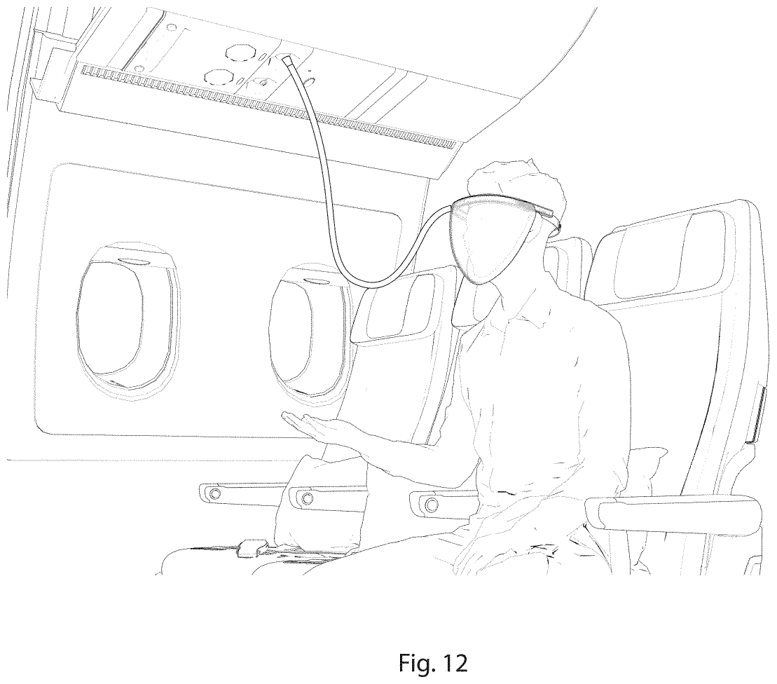

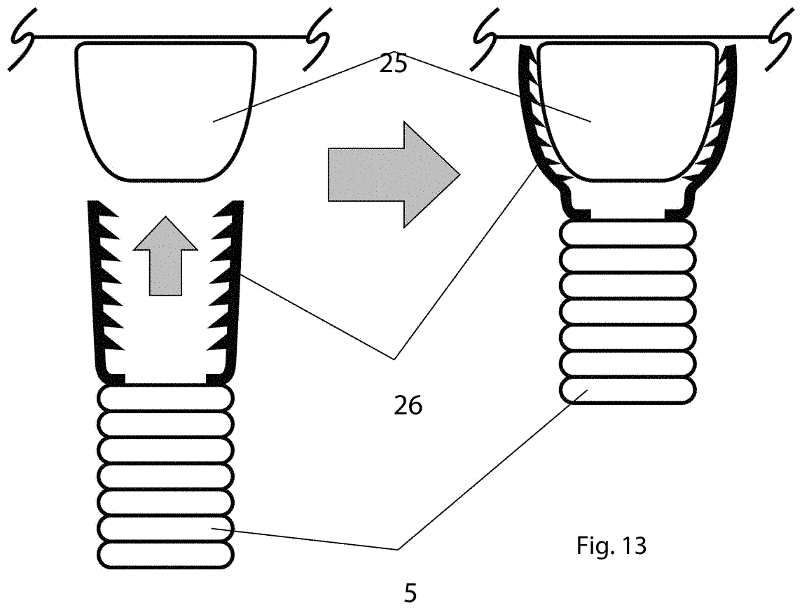

In particular examples, the air filtration face covering device may be integrated into other existing or new forced air filtration systems which can be utilized as a source for blowing clean, filtered air into the visor assembly 11. FIG. 12 is a front-left-side perspective view of a powered air filtration face covering device according to embodiments of the disclosure being utilized in an application where clean, filtered air being forced into the visor assembly is coming from a source other than the electrically powered air filtration blower unit. The source for this filtered air may be from, but not limited to, overhead air receptacles commonly found in airplanes. FIG. 13 illustrates a cross-section of an example coupling mechanism for overhead air receptacles commonly found in airplanes. An example application of this may be, but not limited to, utilizing overhead air receptacles 25 commonly found in airplanes (see, e.g., FIG. 12) to be a source for directing forced filtered air into the claimed air filtration face covering device. As can be seen in FIG. 13, a coupling mechanism 26 may be used to couple an air tube (e.g., air tube 5) to an air receptacle 25 commonly found in airplanes. Various coupling techniques or mechanisms may be used to couple the protective face covering of the present disclosure with the air receptacle of an airplane or other system. Magnets, mechanical coupling devices, and or pressure devices may be used to couple a fluid conduit such as an air hose to the receptacle and the protective face covering.

A method of filtering fluid may comprise using any one of the protective face coverings described herein. As an example, a method may comprise directing a curtain of filter fluid across a face of a wearer and filtering fluid exhausted by the wearer.

The disclosure comprises at least the following aspects:

Aspect 1: A protective face covering comprising: an electrically powered air filtration blower unit; an adjustable head strap with variable size attachment mechanism; and a head mounted hollow air frame; a contoured clear lens/visor with attached fully enclosed membrane barrier.

Aspect 2: The protective face covering of aspect 1, wherein the electrically powered air filtration blower unit comprises one or more removable/or replaceable rechargeable batteries.

Aspect 3: The protective face covering of any one of aspects 1-2, wherein the electrically powered air filtration blower unit comprises one or more removable or replaceable HEPA filters.

Aspect 4: The protective face covering of any one of aspects 1-3, wherein the head strap comprises a variable length elastic loop attachment system.

Aspect 5: The protective face covering of any one of aspects 1-4, wherein the elastic loop attachment system comprises a snap fit hook receptacle.

Aspect 6: The protective face covering of any one of aspects 1-5, wherein the hollow air frame contains one or more air intake ports accepting forced filtered air coming from the powered filter blower unit by utilizing, but not limited to, one or more flexible air tubes to distribute the filtered air.

Aspect 7: The protective face covering of any one of aspects 1-6, wherein the hollow air frame contains a slit opening along the frontal bottom edge of the air frame to create a protective barrier or ‘curtain’ of clean filtered air exhausted downward in front of the wearer's face.

Aspect 8: The protective face covering of any one of aspects 1-7, wherein a contoured clear lens/visor covering the wearer's mouth, nose and eyes is attached to or molded in combination with the frontal portion of the hollow air frame. As an example, the contouring of the lens is offset to closely fit the shape of a standard human face, thus creating a cavity with minimal air volume surrounding the wearer's face.

Aspect 9: The protective face covering of any one of aspects 1-8, wherein an elastomeric flexible membrane is attached to the peripheral or perimeter edge of the lens or visor, creating an air barrier cavity between the visor and the wearer's face. As an example, the membrane may wrap between the head strap and the hollow air frame, and continues to be attached to the top surface of the air frame, thus creating a singular, unobstructed complete barrier for positive air pressure to be contained within the cavity.

Aspect 10: The protective face covering of any one of aspects 1-9, wherein an internal dam wall found inside the hollow air frame adjacent the exhaust slit, directs forced air to exhaust through the slit, and re-enter the hollow air frame in the rearward exhaust intake hole.

Aspect 11: The protective face covering of any one of aspects 1-10, wherein air being forced into the exhaust area of the hollow air frame travels through an exhaust filter, thus filtering exhausted air exhaled by the wearer.

Aspect 12: The protective face covering of any one of aspects 1-11, wherein the visor assembly is connected to the head strap with a rotational pivoting mechanism.

Aspect 13: The protective face covering of any one of aspects 1-12, wherein the rotational pivoting mechanism has defined rotational start and stop points, defining open and closed positions of the visor assembly.

Aspect 14: The protective face covering of any one of aspects 1-13, wherein the defined start and stop positions of the rotational mechanism, and variable positions in between, have multiple position locking functionality.

Aspect 15: The protective face covering of any one of aspects 1-14, wherein the rotational pivot system of the visor assembly is capable of continuing to deliver a curtain of clean, filtered air when the device is rotated in the open upward position, allowing access to the wearer's mouth, nose, eyes and face, while still being protected from inhaling particulate matter from surrounding ambient air.

Aspect 16: The protective face covering of any one of aspects 1-15, containing a portable, powered filtration system that can be worn in a variety of ways, including on the shoulders of the user without the use of straps.

Aspect 17: The protective face covering of any one of aspects 1-16, wherein filtered air coming from an alternative source, such as overhead airline air nozzles, can be utilized to force/blow filtered air into the claimed device.

Aspect 18: The protective face covering of any one of aspects 1-17, further comprising one or more UV-C devices configured to irradiate a fluid passing to or through the hollow air frame.

Aspect 19: A method of filtering fluid comprising directing a curtain of filter fluid across a face of a wearer and filtering fluid exhausted by the wearer.

Aspect 20: A protective face covering comprising: a frame configured to be mounted on a head of a wearer, the frame comprising a main body having a curvilinear shape configured to extend around a forehead of the wearer, wherein at least a portion of the main body is hollow and defines an inlet passage and an exhaust passage therein; a visor coupled to the frame and configured to be disposed adjacent a face of a user in a first position; a membrane barrier coupled to one or more of the visor or the frame and configured to seal against a portion of the wearer to define a sealed cavity between the visor and the face of the wearer in the first position; and a blower unit in fluid communication with the inlet passage, the blower unit configured to provide a volume of filtered fluid to the inlet passage, wherein the frame comprises an inlet aperture disposed adjacent a front portion of the frame and configured to direct a curtain of fluid into the sealed cavity in the first position.

Aspect 21: A protective face covering comprising: a frame configured to be mounted on a head of a wearer, the frame comprising a main body defining an inlet passage and an exhaust passage therein; a visor coupled to the frame and configured to be disposed adjacent a face of a user in a first position; a membrane barrier coupled to one or more of the visor or the frame and configured to seal against a portion of the wearer to define a sealed cavity between the visor and the face of the wearer; and a blower unit in fluid communication with the inlet passage, the blower unit configured to provide a volume of filtered fluid to the inlet passage.

Aspect 22: The protective face covering of any one of aspects 20-21, wherein the frame comprises an inlet aperture disposed adjacent a front portion of the frame and configured to direct a curtain of fluid into the sealed cavity in the first position.

Aspect 23: The protective face covering of any one of aspects 20-22, wherein the blower unit is a powered unit.

Aspect 24: The protective face covering of any one of aspects 20-23, wherein the blower unit is powered by one or more batteries.

Aspect 25: The protective face covering of any one of aspects 20-24, wherein the blower unit comprises a HEPA filter configured to filter fluid being provided to the inlet passage.

Aspect 26: The protective face covering of any one of aspects 20-25, further comprising a fluid conduit extending between the blower unit and the frame.

Aspect 27: The protective face covering of any one of aspects 20-26, further comprising one or more UV-C devices configured to irradiate a fluid passing through the fluid conduit with UV-C spectrum light.

Aspect 28: The protective face covering of any one of aspects 20-27, wherein the fluid conduit is flexible.

Aspect 29: The protective face covering of any one of aspects 20-28, wherein the membrane barrier is coupled to a peripheral edge of the visor.

Aspect 30: The protective face covering of any one of aspects 20-29, further comprising an internal dam wall disposed inside the inlet passage and adjacent the inlet aperture, wherein the internal dam wall operates to direct forced air to flow through the inlet aperture.

Aspect 31: The protective face covering of any one of aspects 20-30, further comprising an internal dam wall disposed inside the inlet passage and adjacent the inlet aperture, wherein the internal dam wall operates to direct forced air to flow through the inlet aperture, through the sealed cavity, and to the exhaust passage.

Aspect 32: The protective face covering of any one of aspects 20-31, further comprising an exhaust filter disposed in the exhaust passage and configured to filter fluid passing from the sealed cavity through the exhaust passage.

Aspect 33: The protective face covering of any one of aspects 20-32, wherein the visor is configured to rotationally pivot to a second position.

Aspect 34: The protective face covering of any one of aspects 20-33, wherein the frame comprises an inlet aperture disposed adjacent a front portion of the frame and configured to direct a curtain of fluid into the sealed cavity in the first position, and wherein the visor is configured to rotationally pivot to a second position without impeding the curtain of fluid.

Aspect 35: A protective face covering comprising: a frame configured to be mounted on a head of a wearer, the frame comprising a main body having a curvilinear shape configured to extend around a forehead of the wearer, wherein at least a portion of the main body is hollow and defines an inlet passage and an exhaust passage therein; a visor rotatably coupled to the frame and configured to be disposed adjacent a face of a user in a closed position and rotated about the frame into a second position; and a blower unit in fluid communication with the inlet passage, the blower unit configured to provide a volume of filtered fluid to the inlet passage, wherein the frame comprises an inlet aperture disposed adjacent a front portion of the frame and configured to direct a curtain of fluid across the face of the wearer.

Aspect 36: The protective face covering of any one of aspect 35, wherein the blower unit is comprised as part of an air handling system.

Aspect 37: The protective face covering of any one of aspects 35-36, wherein the blower unit is comprised as part of an air handling system of an airplane.

Aspect 38: The protective face covering of any one of aspects 35-37, further comprising a coupling mechanism and a fluid conduit configured to couple the blower unit to the inlet aperture.

While embodiments of the disclosure have been described and disclosed in certain terms and has disclosed certain embodiments or modifications, persons skilled in the art who have acquainted themselves with the disclosure, will appreciate that it is not necessarily limited by such terms, nor to the specific embodiments and modification disclosed herein. Thus, a wide variety of alternatives, suggested by the teachings herein, can be practiced without departing from the spirit of the disclosure, and rights to such alternatives are particularly reserved and considered within the scope of the disclosure.

What is claimed is:

1. A protective face covering comprising:
a frame configured to be mounted on a head of a wearer, the frame comprising a main body having a curvilinear shape configured to extend around a forehead of the wearer, wherein at least a portion of the main body is hollow and defines an inlet passage and an exhaust passage therein;
a visor coupled to the frame and configured to be disposed adjacent a face of a user in a first position;
a membrane barrier coupled to one or more of the visor or the frame and configured to seal against a portion of the wearer to define a sealed cavity between the visor and the face of the wearer in the first position; and
a blower unit in fluid communication with the inlet passage, the blower unit configured to provide a volume of filtered fluid to the inlet passage,
wherein the frame comprises an inlet aperture disposed adjacent a front portion of the frame and configured to direct a curtain of fluid into the sealed cavity in the first position.

2. A protective face covering comprising:
a frame configured to be mounted on a head of a wearer, the frame comprising a main body defining an inlet passage and an exhaust passage therein;
a visor coupled to the frame and configured to be disposed adjacent a face of a user in a first position;
a membrane barrier coupled to one or more of the visor or the frame and configured to seal against a portion of the wearer to define a sealed cavity between the visor and the face of the wearer; and
a blower unit in fluid communication with the inlet passage, the blower unit configured to provide a volume of filtered fluid to the inlet passage.

3. The protective face covering of claim 2, wherein the frame comprises an inlet aperture disposed adjacent a front portion of the frame and configured to direct a curtain of fluid into the sealed cavity in the first position.

4. The protective face covering of claim 2, wherein the blower unit is a powered unit.

5. The protective face covering of claim 4, wherein the blower unit is powered by one or more batteries.

6. The protective face covering of claim 2, wherein the blower unit comprises a HEPA filter configured to filter fluid being provided to the inlet passage.

7. The protective face covering of claim 2, further comprising a fluid conduit extending between the blower unit and the frame.

8. The protective face covering of claim 7, further comprising one or more UV-C devices configured to irradiate a fluid passing through the fluid conduit with UV-C spectrum light.

9. The protective face covering of claim 7, wherein the fluid conduit is flexible.

10. The protective face covering of claim 2, wherein the membrane barrier is coupled to a peripheral edge of the visor.

11. The protective face covering of claim 3, further comprising an internal dam wall disposed inside the inlet passage and adjacent the inlet aperture, wherein the internal dam wall operates to direct forced air to flow through the inlet aperture.

12. The protective face covering of claim 3, further comprising an internal dam wall disposed inside the inlet passage and adjacent the inlet aperture, wherein the internal dam wall operates to direct forced air to flow through the inlet aperture, through the sealed cavity, and to the exhaust passage.

13. The protective face covering of claim 2, further comprising an exhaust filter disposed in the exhaust passage and configured to filter fluid passing from the sealed cavity through the exhaust passage.

14. The protective face covering of claim 2, wherein the visor is configured to rotationally pivot to a second position.

15. The protective face covering of claim 2, wherein the frame comprises an inlet aperture disposed adjacent a front portion of the frame and configured to direct a curtain of fluid into the sealed cavity in the first position, and wherein the visor is configured to rotationally pivot to a second position without impeding the curtain of fluid.

16. A protective face covering comprising:
a frame configured to be mounted on a head of a wearer, the frame comprising a main body having a curvilinear shape configured to extend around a forehead of the wearer, wherein at least a portion of the main body is hollow and defines an inlet passage and an exhaust passage therein;
a visor rotatably coupled to the frame and configured to be disposed adjacent a face of a user in a closed position and rotated about the frame into a second position; and
a blower unit in fluid communication with the inlet passage, the blower unit configured to provide a volume of filtered fluid to the inlet passage,
wherein the frame comprises an inlet aperture disposed adjacent a front portion of the frame and configured to direct a curtain of fluid across the face of the wearer.

17. The protective face covering of claim 16, wherein the blower unit is comprised as part of an air handling system.

18. The protective face covering of claim 16, wherein the blower unit is comprised as part of an air handling system of an airplane.

19. The protective face covering of claim 18, further comprising a coupling mechanism and a fluid conduit configured to couple the blower unit to the inlet aperture.
